(19) United States
(12) Patent Application Publication
(10) Pub. No.: US 2009/0017145 A1
(43) Pub. Date: Jan. 15, 2009

(54) Tri ascorbate formulation

(76) Inventor: Wayne Gorsek, Boynton Beach, FL (US)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(21) Appl. No.: 11/822,751

(22) Filed: Jul. 10, 2007

(51) Int. Cl. A61K 31/375; A61K 36/185; A61K 36/73; A61P 17/18; A61P 19/02; A61P 37/00; A61P 9/00

(52) U.S. Cl. 424/765; 424/725; 514/474

(57) ABSTRACT

An oral formulation that is a combination of three mineral ascorbates and metabolites for antioxidant protection against free radical damage, healthy skin maintenance, support for vision health, healthy joint support, cardiovascular support, and maintenance of the immune system.

### BACKGROUND OF THE INVENTION

The present invention relates to a novel therapeutic use of a combination of three mineral ascorbates and metabolites for antioxidant protection against free radical damage, healthy skin maintenance, support for vision health, healthy joint support, cardiovascular support, and maintenance of the immune system.

More specifically, the present invention relates to the coordinated use of calcium ascorbate, magnesium ascorbate and zinc ascorbate in a formulation. In addition to the ascorbate components there is present calcium threonate, calcium xylonate, and calcium lyxonate.

### SUMMARY OF THE INVENTION

Therefore, the present invention also relates to orally administrable pharmaceutical compositions suitable for preventing oxygen and free radicals from damaging body tissue. The formulation is necessary for the production of proteins, collagen and elastin, which strengthen arteries, bones, teeth, cartilage, and other tissues. It helps the body absorb iron, helps lower blood pressure and blood cholesterol, and reduces the effects of some allergy-producing substances.

The invention described herein is based on the surprising synergistic effect occurring between the combination of the three ascorbates, calcium, magnesium and zinc. The addition of the three calcium component compounds provides for a uniques and superior oral formulation. This synergistic effect is particularly surprising.

### DETAILED DESCRIPTION OF THE INVENTION

It has now been found that the combined unique ascorbate tri formulation of calcium ascorbate, magnesium ascorbate not only prevents cell damage but also enhances the process of cell repair, thus allowing remarkable therapeutic results to be achieved.

Further, there is added to the unique ascorbate tri formulation calcium threonate, calcium xylonite, and calcium lyxonate.

The unique ascorbate tri formulation is an excellent reducing agent, and it acts as a cofactor in various biochemical reactions to reduce the transition metals, iron and copper.

Ascorbate can be oxidized by most reactive oxygen and nitrogen species thought to play roles in tissue injury associated with various diseases. These species include superoxide, hydroxyl, peroxyl and nitroxide radicals, as well as such non-radical reactive species as singlet oxygen, peroxynitrite and hypochlorite. By virtue of this scavenging activity, ascorbate inhibits lipid peroxidation, oxidative DNA damage and oxidative protein damage.

Ascorbate is oxidized by reactive oxygen and nitrogen species to the semidehydroascorbate radical that is either reconverted to ascorbate via the enzyme NADH semidehydroascorbate reductase or is converted to dehydroascorbate

Dehydroascorbate in turn can be converted back to ascorbate via glutathione-dependent enzymes or catabolized.

Ascorbate can act as a secondary antioxidant. At least in vitro, ascorbate regenerates the major lipid antioxidant alpha-tocopherol from the alpha-tocopheroxyl radical form. Ascorbate may also participate in regenerating and sparing alpha-tocopherol in vivo, though this has not been clearly demonstrated. The unique ascorbate tri formulation does preserve intracellular reduced glutathione concentrations.

As noted above, Unique ascorbate tri formulation helps preserve intracellular reduced glutathione concentrations. This activity likely helps maintain nitric oxide levels and potentiates its vasoactive effects. Oral Unique ascorbate tri formulation can reach high enough concentrations intracellularly to scavenge superoxide radicals. Thus, intracellular sources of superoxide that impair nitric oxide may be scavenged by oral Unique ascorbate tri formulation. Recently, it has been found that ascorbic acid enhances nitric oxide synthase activity by increasing intracellular tetrahydrobiopterin.

The unique ascorbate tri formulation may modulate prostaglandin synthesis to favor the production of eicosanoids with antithrombotic and vasodilatory activity. The possible sparing and regeneration of alpha-tocopherol by the unique ascorbate tri formulation could be yet another factor in the vitamin's possible anti-atherogenic action.

Evidence appears to suggest that the unique ascorbate tri formulation may have cancer-preventive activity, at least for certain types of cancer. A recent cell-culture study of human breast carcinoma lines may suggest the unique ascorbate tri formulation to improve the antineoplastic activity of doxorubicin, cisplatin and paclitaxel. The mechanism of the effect may be pro-oxidant, not antioxidant, activity of the vitamin in potentiating the effects of these chemotherapeutic agents. Another study suggests that the pro-oxidant form of the unique ascorbate tri formulation may upregulate some of the enzymes involved in DNA repair. This possible activity may play some anticarcinogenic role.

The unique ascorbate tri formulation may have anti-hypertensive activity in some. The mechanism of this possible effect is a matter of speculation. As observed above, the unique ascorbate tri formulation may help maintain nitric oxide levels and potentiate its vasoactive effects. There is an indication that the unique ascorbate tri formulation may improve endothelial-dependent vasodilation in those with essential hypertension, as well as in those with hypercholesterolemia, and may help restore nitric oxide-mediated flow-dependent vasodilation in those with congestive heart failure.

There is some evidence that the unique ascorbate tri formulation supplementation decreases the incidence, severity and duration of common cold symptoms in some. It is thought that this is due, at least in part, to antihistaminic activity of the unique ascorbate tri formulation.

Some studies suggest a protective effect of the unique ascorbate tri formulation supplementation against cataracts. Age-related lens opacities are thought to be due to oxidative stress.

The unique ascorbate tri formulation may protect against asthma and other obstructive pulmonary diseases, as well as protect the airways against the effects of allergens, viral infections and irritants in some. Allergens, viruses and irritants, including ozone, nitrogen oxides and sulfur oxides, subject the airways to increased oxidative stress, which can lead to bronchoconstriction. The possible protective action of the unique ascorbate tri formulation appears clearly due to its antioxidant properties.

The antioxidant properties of the unique ascorbate tri formulation can also account for its role in protecting against the tissue-damaging effect of some toxic chemicals and heavy metals. High serum levels of ascorbic acid have been reported to be associated with a decreased prevalence of elevated blood lead levels. One study compared the chelating properties of ascorbic acid and the known lead-chelating agent EDTA and found them to have equivalent activity with respect to lead.

The formulation comprises:

| | | |
|---|---|---|
| Tri Formulation Blend | 10-100,000 | mg |
| Calcium (as Calcium Ascorbate and | 1-10,000 | mg |
| Calcium Threonate)) | | |
| Citrus Bioflavanoids Complex | 2-20,000 | mg |
| Rosehips Extract | 0.25-2,500 | mg |
| Rutin | 0.25-2,500 | mg |
| Acerola Cherry Extract | 0.19-1,900 | mg |

| Tri Formulation Blend | Dosage Range | |
|---|---|---|
| Calcium Ascorbate | 9.20-920,000 | mg |
| Magnesium Ascorbate | 0.4925-49,250 | mg |
| Zinc Ascorbate | 0.0075-7.5 | mg |
| Calcium Threonate | 0.10-10,000 | mg |
| Calcium Xylonate | 0.10-10,000 | mg |
| Calcium Lyxonate | 0.10-10,000 | mg |

The preferred formulation comprises:

| | | % Concentration of | | | |
|---|---|---|---|---|---|
| | | Ascorbic Acid/ | | Amount of Ascorbic | Amount of |
| Tri Formulation Blend | Dosage | Mineral | | Acid | Mineral |
| Calcium Ascorbate | 920 | mg. | 80%/9% | 736 mg. | 82.8 mg. |
| Magnesium Ascorbate | 49.25 | mg. | 80%/6.5% | 39.4 mg. | 3.20 mg. |
| Zinc Ascorbate | 0.75 | mg. | 80%/8% | 0.6 mg. | 0.06 mg. |
| Calcium Threonate | 10 | mg. | 50%/50% | 5 mg. of L-Threonate | 5 mg. of Calcium |
| Calcium Xylonate | 10 | mg. | 50%/50% | 2.5 mg. of L-Xylonate | 2.5 mg. of Calcium |
| Calcium Lyxonate | 10 | mg. | 50%/50% | 2.5 mg. of L-Lyxonate | 2.5 mg. of Calcium |

Tri Ascorbate Formulation Complex 1000 mg. Tablets
Supplement Facts
Serving Size: 1 Tablet
Servings per Bottle: 300 servings

| | | | | Raw Mat. |
|---|---|---|---|---|
| | | | % | (mg.) 2% |
| Amount Per Serving | Dosage | | Concentration | overage |
| Tri Formulation Blend | 1000 | mg. | 78% | 1275.0 mgs. |
| Calcium (as Calcium | 100 | mg. | 9% | |
| Ascorbate and Calcium | | | | |
| Threonate)) | | | | |
| Citrus Bioflavanoids | 200 | mg. | 100% | 204.0 mgs. |
| Complex | | | | |
| Rosehips Extract | 25 | mg. | 100% | 25.5 mgs. |
| Rutin | 25 | mg. | 100% | 25.5 mgs. |
| Acerola Cherry Extract | 19 | mg. | 100% | 19.4 mgs. |
| | | | | 1549.4 mg. |

Weight of Active Ingredients per Tablet: 1,549.40 mgs.
Approximate Weight of Finished Tablet: 1700.00 mgs.
Other Ingredients: Di-Calcium Phophate, Cellulose, Stearic Acid, Magnesium Stearate, Silica, Pharmaceutical Glaze

Calcium Ascorbate is an optimal combination of Ascorbic Acid (Vitamin C) with Calcium Carbonate
Magnesium Ascorbate is an optimal combination of Ascorbic Acid (Vitamin C) with Magnesium Carbonate
Zinc Ascorbate is an optimal combination of Ascorbic Acid (Vitamin C) with Zinc Carbonate
Calcium threonate, calcium xylonite and calcium lyxonate are metabolites that are natural components that exist in plants and animals.

What is claimed is:

1. A Tri Ascorbate oral composition comprising:
An effective amount of Calcium ascorbate, magnesium ascorbate, zinc ascorbate, calcium threonate, calcium xylonite and calcium lyxonate.

2. The composition of claim 1, further comprising:
Calcium as Calcium Ascorbate, calcium threonate, Citrus bioflavanoids complex, rosehips extract, rutin and acerola extract.

3. The composition of claim 2, further comprising:
Di-calcium phosphate, cellulose, stearic acid, magnesium stearate, silica and pharmaceutical glaze.

4. A Tri Ascorbate oral composition comprising:
A tri-component blend of Calcium ascorbate 9.20-920,000 mg;
Magnesium ascorbate 0.4925-49,250 mg;
Zinc ascorbate 0.0075-7.5 mg;
(Calcium as Calcium Ascorbate, calcium threonate) 1.0-100,00 mg.
Calcium threonate 0.10-10,000 mg.;
Calcium xylonite 0.10-10,000 mg.; and
Calcium lyxonate 0.10-10,000 mg

5. The composition of claim 4, further comprising:
A group of inactive components comprising Di-calcium phosphate, cellulose, stearic acid, magnesium stearate, silica and pharmaceutical glaze 1.50-150,000 mg.
